(19) United States
(12) Patent Application Publication
(10) Pub. No.: US 2019/0133460 A1
(43) Pub. Date: May 9, 2019

(54) BLOOD PRESSURE MONITORING SYSTEM WITH ISOLATION

(71) Applicant: Edwards Lifesciences Corporation, Irvine, CA (US)

(72) Inventors: Jason A. Wine, Placentia, CA (US); Alexander H. Siemons, Yorba Linda, CA (US); Siddarth Kamath Shevgoor, Laguna Beach, CA (US)

(21) Appl. No.: 16/175,718

(22) Filed: Oct. 30, 2018

Related U.S. Application Data
(60) Provisional application No. 62/583,977, filed on Nov. 9, 2017.

(51) Int. Cl. A61B 5/0215; A61B 5/15; A61B 5/00; A61B 5/0205

(52) U.S. Cl. CPC A61B 5/0215; A61B 5/150992; A61B 5/02141; A61B 5/0205; A61B 5/725

(57) ABSTRACT

Disclosed is an integrated blood sampling-pressure monitoring system to sample blood from a patient and to measure the blood pressure of the patient. The integrated blood sampling-pressure monitoring system may include: a pressure transducer to measure the blood pressure of the patient; a blood sampling portion to sample blood from the patient; and a restrictor. The restrictor may be interposed between the pressure transducer and the blood sampling portion, such that, the pressure transducer may be located closer to the patient than the blood sampling portion along a fluid line when the blood sampling-pressure monitoring system is connected to the patient.

### CROSS-REFERENCE TO RELATED APPLICATIONS

This application claims priority to U.S. Provisional Application No. 62/583,977, filed Nov. 9, 2017, the contents of which are incorporated herein by reference.

### BACKGROUND

### Field

Embodiments of the invention relate to blood sampling systems and, in particular, to closed blood sampling systems with a clearing reservoir and blood pressure monitoring.

### Relevant Background

A blood pressure monitoring system comprising a pressure transducer (e.g., a disposable pressure transducer “DPT”) may be used to continuously measure a patient's blood pressure. This type of system may be composed of a patient tubing connection (which is typically attached to an arterial line or pulmonary artery catheter “PAC”), flexible tubing, and an integral DPT. The tubing is filled with saline and is attached to the patient's artery or vein. The DPT is preferably positioned at the same height as the phlebostatic axis of the patient, and the blood pressure is measured through the tubing system.

A means to sample blood (e.g., a blood sampling system), such as a Venous Arterial blood Management Protection (VAMP) system, is often included in or integrated with the pressure transducer system. A blood sampling system may be composed of a reservoir and a sampling site, allowing the sampling of blood through an access port in the tubing system. The reservoir houses a blood-saline mixture (or “clearing volume”) when opened, allowing blood to be sampled from the integral sampling site. After all samples are taken, the clearing volume is infused back into the patient, preventing the loss of blood in critically ill patients.

The mechanical elements which aid in the usability of the blood sampling system (including long flexible tubing, reservoirs, sampling sites, etc.) fundamentally diminish the accuracy of the blood pressure monitoring system. As the natural frequency of the system decreases, the ability of the system to faithfully reproduce the frequencies included within the patient blood pressure waveform decreases. This can have a significant effect on the reported blood pressure values.

### SUMMARY

Embodiments of the invention may relate to an integrated blood sampling-pressure monitoring system to sample blood from a patient and to measure the blood pressure of the patient. The integrated blood sampling-pressure monitoring system may include: a pressure transducer to measure the blood pressure of the patient; a blood sampling portion to sample blood from the patient; and a restrictor. The restrictor may be interposed between the pressure transducer and the blood sampling portion, such that, the pressure transducer may be located closer to the patient than the blood sampling portion along a fluid line when the blood sampling-pressure monitoring system is connected to the patient.

### BRIEF DESCRIPTION OF THE DRAWINGS

FIG. 1 is a perspective view of an example existing blood sampling system.

FIG. 2 is a diagram illustrating an example existing integrated blood sampling-pressure monitoring system.

FIG. 3 is a block diagram illustrating an example integrated blood sampling-pressure monitoring system, according to one embodiment of the invention.

FIG. 4 is another diagram illustrating an example integrated blood sampling-pressure monitoring system, according to one embodiment of the invention.

FIG. 5 is a block diagram illustrating an example integrated blood sampling-pressure monitoring system, according to one embodiment of the invention.

FIG. 6 is a diagram illustrating a cross-section view of a double lumen tube used in the system of FIG. 5.

FIG. 7 is a block diagram illustrating another example integrated blood sampling-pressure monitoring system, according to one embodiment of the invention.

FIGS. 8A and 8B are diagrams illustrating cross-section views of an example restrictor.

FIG. 9 is a diagram illustrating a cross-section view of another example restrictor.

FIG. 10 is a diagram illustrating a cross-section view of yet another example restrictor.

### DETAILED DESCRIPTION

Embodiments of the invention are related to an integrated blood sampling-pressure monitoring system in which the blood sampling portion and the blood pressure monitoring portion are isolated with respect to fluid pressure transmission by a restrictor member. Although pressure isolated, the two portions are still hydraulically connected for flushing and patency.

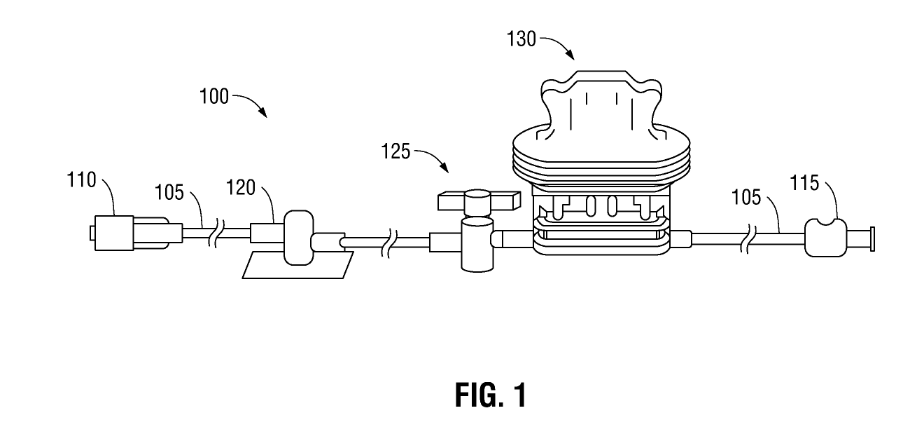

Referring to FIG. 1, a perspective view of an example existing blood sampling system 100 is shown. The system 100 may be a VAMP system. The system 100 may include a fluid line 105 having a distal end 110 and a proximal end 115 relative to the clinician. The system 100 may also include a blood sampling site 120 (e.g., a blood sampling device), a stopcock valve 125, and a reservoir 130. The distal end 110 of the fluid line 105 is coupled to an intravenous (IV) needle that is inserted into a patient's vein or artery. The proximal end 115 of the fluid line 105 is coupled to pressure monitoring lines and/or continuous IV infusion (or saline) line.

In the quiescent state, the stopcock valve 125 is open, allowing solution from the IV assembly to be fed through the fluid line 105 and the IV needle into the patient. To obtain a blood sample, the reservoir 130 is slowly moved to an open position (e.g., by pulling a plunger), allowing blood to flow upstream past the sampling site 120 and into the reservoir 130. The stopcock valve 125, located downstream from the reservoir, is then placed in a closed position preventing IV fluid (or saline) from entering the blood sampling site 120. Then, a syringe is positioned through the blood sampling site 120 to draw blood from the patient. After the sample is drawn from the patient, the syringe is detached from the blood sampling site 120, and the stopcock valve 125 is moved to an open position. The reservoir 130 is slowly returned to a closed position (e.g., by pushing the plunger), infusing the blood-IV fluid (saline) mixture (the clearing volume) in the reservoir 130 back into the patient and reestablishing the connection between the patient's circulatory system and the IV infusion or saline line.

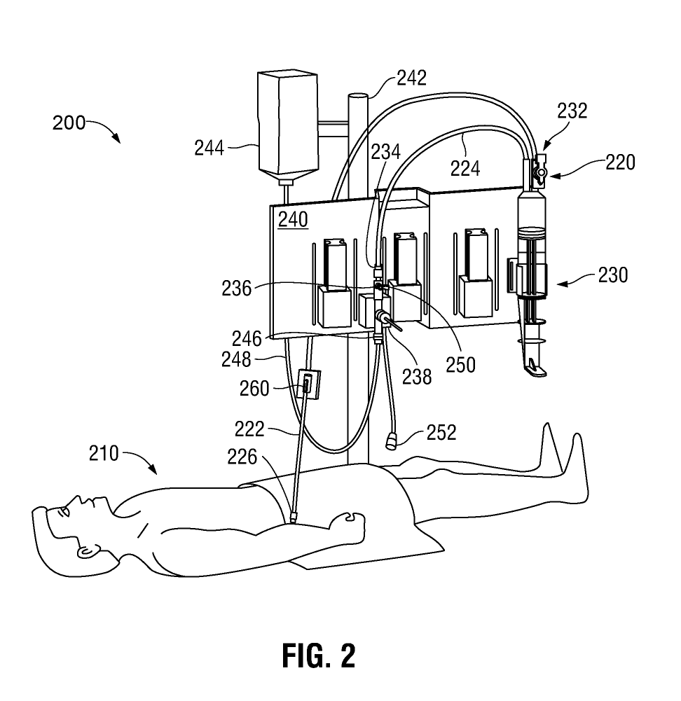

Referring to FIG. 2, a diagram illustrating an example existing integrated blood sampling-pressure monitoring system 200 is shown. The system 200 is shown in the environment of a typical set up in a hospital room and connected to a patient 210. The system 200 comprises a fluid line having a distal segment 222 toward the patient and a proximal segment 224. The fluid line is primarily medical grade pressure tubing. The distal segment 222 may terminate in a male luer connector 226 for attaching to a female luer connector (not shown) of an injection site, or other conduit leading to the patient. A reservoir 230 connects to the proximal segment 224 of the fluid line, and to the distal segment 222 of the fluid line via a stopcock valve 232. The system 200 further comprises a blood sampling site 260.

The proximal segment 224 extends from the reservoir 230 and terminates in a female luer connector 234 attached to a stopcock valve 236 of a pressure transducer 238. The reservoir 230 and pressure transducer 238 removably mount to a bracket 240 which, in turn, may be secured to a conventional pole support 242 with the reservoir in a vertical orientation.

The blood sampling system 220 forms a portion of the integrated system 200. The fluid pressure transducer 238 may be a DPT. A supply of flush solution 244 connects to a flush port 246 of the transducer 238 via tubing 248. Typically, the flush solution 244 comprises a bag of physiological fluid such as saline surrounded by a pressurized sleeve that squeezes the fluid and forces it through the tubing 248. In addition, an IV infusion fluid supply (not shown) may be provided in communication with an infusion port 250 of the stopcock valve 236. The pressure transducer 238 is thus placed in fluid communication with the arterial or venous system of the patient through the fluid line, and may include a cable and plug 252 to connect to a suitable display monitor (not shown).

Therefore, in an existing, conventional integrated blood sampling-pressure monitoring system, the pressure transducer is typically located upstream from the blood sampling portion of the system and away from the patient. As described above, the mechanical elements of the blood sampling portion of the system which aid in the usability of the system fundamentally diminish the accuracy of the pressure monitoring system. As the natural frequency of the system decreases, the ability of the system to faithfully reproduce the frequencies included within the patient blood pressure waveform decreases. This can have a significant effect on the reported blood pressure values.

Embodiments of the invention are related to an integrated blood sampling-pressure monitoring system in which the blood sampling portion and the blood pressure monitoring portion are isolated with respect to fluid pressure transmission by a restrictor member. Although pressure isolated, the two portions are still hydraulically connected for flushing and patency.

Further, according to embodiments of the invention, the pressure transducer may be located downstream from the blood sampling portion (e.g., in the distal segment of the fluid line) of the system and be closer to the patient. A restrictor may be placed between the pressure transducer and the blood sampling portion of the system, allowing the hydraulic connection between the two portions of the system for flushing and patency, but isolating the two portions from each other with respect to pressure transmission.

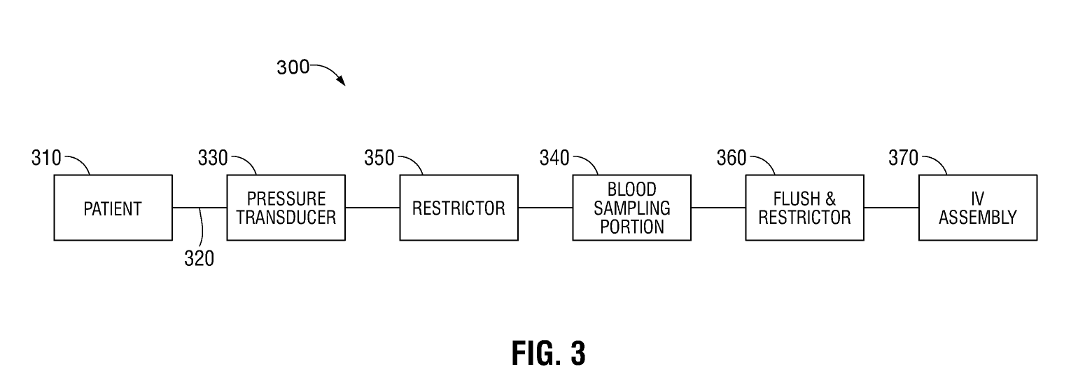

Referring to FIG. 3, a block diagram illustrating an example integrated blood sampling-pressure monitoring system 300 to sample blood from a patient 310 and to measure the blood pressure of the patient 310, according to one embodiment of the invention, is shown. The integrated blood sampling-pressure monitoring system 300 may include: a pressure transducer 330 to measure the blood pressure of the patient 310; a blood sampling portion 340 to sample blood from the patient 310; and a restrictor 350. The restrictor 350 may be interposed between the pressure transducer 330 and the blood sampling portion 340, such that, the pressure transducer 330 may be located closer to the patient 310 than the blood sampling portion 340 along a fluid line 320 when the blood sampling-pressure monitoring system is connected to the patient.

As an example, the system 300 may connected to the patient 310's vein or artery via a fluid line 320. The fluid line 320 also connects the various components of the system 300. The system 300 includes the pressure transducer 330 (e.g., a DPT) in the distal segment of the fluid line 320 downstream from the blood sampling portion 340 (e.g., a VAMP system) of the system. In other words, the pressure transducer 330 is closer to the patient 310 than the blood sampling portion 340 of the system. Preferably the restrictor 350 is interposed between the pressure transducer 330 and the blood sampling portion 340 of the system, allowing the hydraulic connection between the two portions of the system for flushing and patency, but isolating the two portions from each other with respect to pressure transmission. In one embodiment, the restrictor 350 may be part of the pressure transducer 330 assembly. Upstream from the blood sampling portion 340 are flush solution and restrictor 360 and an IV assembly 370, which may be similar to the same components in an existing system.

In one embodiment, the tubing between the patient 310 and the pressure transducer 330 may be approximately 30 to 40 inches long, and the tubing between the pressure transducer 330 and the blood sampling portion 340 may be approximately 80 to 100 inches long. In other words, because pressure transmission between the two portions of the system 300 is isolated from each other, longer, softer, and/or more flexible tubing may be used for the blood sampling portion 340 of the system 300 without negatively affecting the accuracy of blood pressure measurements by the pressure transducer 330.

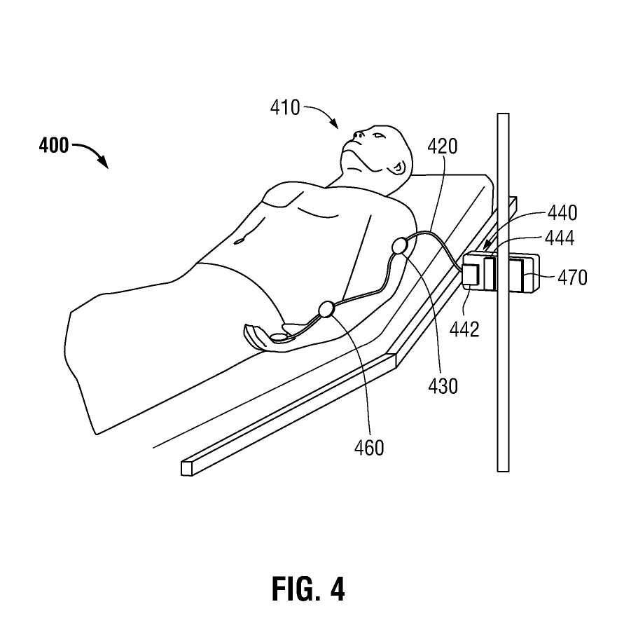

Referring to FIG. 4, another diagram illustrating an example integrated blood sampling-pressure monitoring system 400, according to one embodiment of the invention, is shown. The system 400 is shown in the environment of a typical set up in a hospital room and the system is connected to a patient 410's vein or artery via a fluid line 420. The fluid line 420 also connects the various components of the system 400. A pressure transducer 430 (e.g., a DPT) is located closer to the patient 410 downstream from the blood sampling portion 440 (e.g., a VAMP system) of the system 400, which may comprise a sampling site 442 and a reservoir 444 (e.g., as previously described). A restrictor that allows the hydraulic connection between the pressure transducer 430 and the blood sampling portion 440, but isolates the two portions from each other with respect to pressure transmission may be interposed between the pressure transducer 430 and the blood sampling portion 440, as has and will be described. In one embodiment, the restrictor may be part of the pressure transducer 430 assembly. In one embodiment, an additional sampling site 460 may be provided downstream from the pressure transducer 430. The number of sampling sites does not limit the invention. In different embodiments, either one or both of the sampling sites 442, 460 may be present. A conventional pull-tab and restrictor assembly 470 may be provided upstream from the blood sampling portion 440.

Therefore, in one embodiment, the pressure monitoring portion of the system 400 (comprising the part of the system downstream from the pressure transducer 430 including the pressure transducer 430) may be shortened to arm-length. Thus, the pressure transducer 430 may be fixed on the patient 410 near the phlebostatic axis. Moreover, utilizing the restrictor allows the hydraulic connection between the pressure transducer 430 and the blood sampling portion 440, but isolates the two portions from each other with respect to pressure transmission, the tubing for the blood sampling portion 440 of the system 400 may be adjustable without negatively impacting the accuracy of blood pressure measurements. In other words, longer, softer, and/or more flexible tubing than used in an existing system may be used in the blood sampling portion 440.

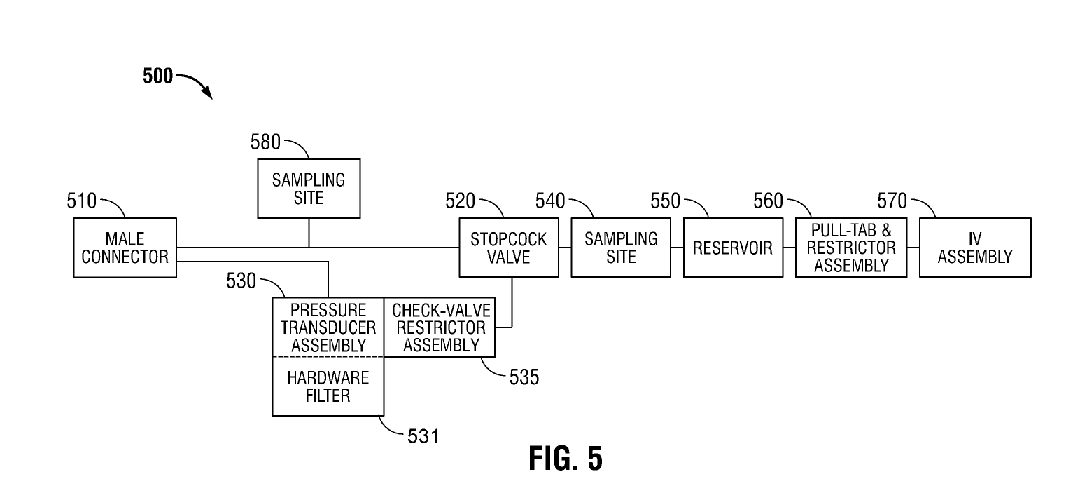

Referring to FIG. 5, a block diagram illustrating an example integrated blood sampling-pressure monitoring system 500, according to one embodiment of the invention, is shown. In the distal segment (toward the patient), the system 500 may terminate with a male luer connector 510. Upstream from the male luer connector 510, the male luer connector 510 is connected to a stopcock valve 520 and a pressure transducer assembly 530 with double lumen tubing (explained in further detail below), each lumen corresponding to either one of the stopcock valve 520 and the pressure transducer assembly 530, wherein the two lumens are isolated from each other. The pressure transducer assembly 530 may comprise a zero-stopcock, and may further comprise, in the same assembly, a check-valve/restrictor assembly 535. Located upstream from the stopcock valve 520 are a sampling site 540, a reservoir 550, a pull-tab and restrictor assembly 560, and an IV assembly 570. It should be appreciated that the stopcock valve 520, sampling site 540, and reservoir 550 constitute a blood sampling portion of the system 500. An additional sampling site 580 may be provided between the male luer connector 510 and the stopcock valve 520. The number of sampling sites does not limit the invention. In different embodiments, either one or both of the sampling sites 540, 580 may be present.

In one embodiment, the length of tubing between the male luer connector 510 and the pressure transducer assembly 530 may be fixed, and may be approximately 40 inches. In other words, the pressure transducer 530 may be mounted inline at a fixed distance from the patient for all kits. In one embodiment, the pressure transducer 530 may be fixed on the patient near the phlebostatic axis. Since the length of tubing between the patient and the pressure transducer assembly 530 is constant, a hardware filter 531 may be included in the pressure transducer assembly 530 (e.g., at a cable connector), that may be utilized to remove all distortions. With the assistance of the hardware filter, soft and compliant tubing may be used for connecting the male luer connector 510 to the pressure transducer assembly 530 and the stopcock valve 520.

The check-valve/restrictor assembly 535 may isolate the pressure transducer 530 from distortions due to the blood sampling portion of the system 500. In one embodiment, if needed, the check-valve/restrictor assembly 535 may allow an approximately 3 milliliters/hour (mL/hr) flow through the pressure transducer system. The pressure transducer system can be primed or flushed. To prime or flush the pressure transducer system, the stopcock valve 520 may be adjusted to direct all flow through the check-valve restrictor 535 and pressure transducer assembly 530.

With the assistance of the check-valve/restrictor assembly 535, soft and compliant materials may be used for the tubing in the blood sampling portion of the system 500 without negatively affecting the accuracy of blood pressure measurements. Moreover, the length of the tubing in the blood sampling portion of the system 500 may be completely flexible. In other words, longer tubing may be used in this portion.

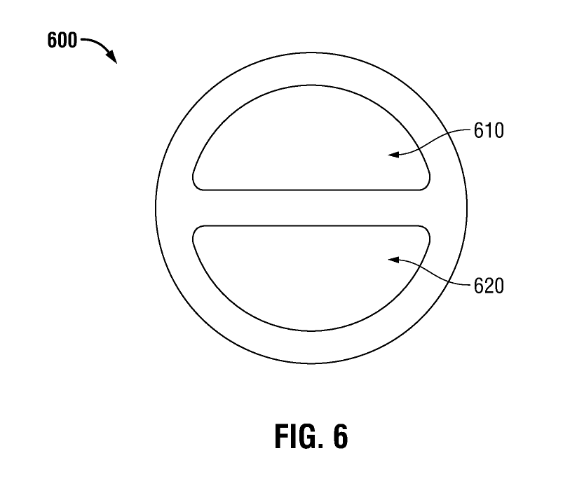

Referring to FIG. 6, a diagram illustrating a cross-section view of a double lumen tube 600 used in the system 500 of FIG. 5 is shown. The double-lumen tube 600 comprises two lumens 610, 620. The cross section of each of the lumens may be semi-circular in shape. Further, the two lumens 610, 620 are separated by impermeable tubing material. Therefore, liquid pressure in the two lumens are isolated.

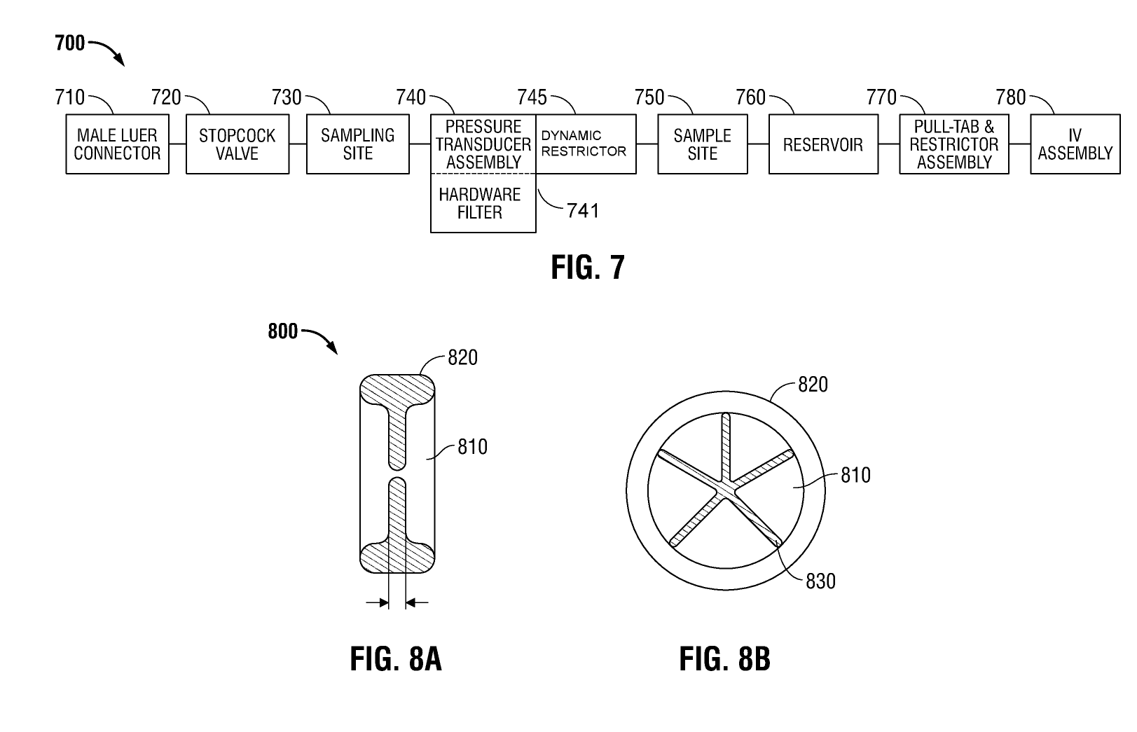

Referring to FIG. 7, a block diagram illustrating another example integrated blood sampling-pressure monitoring system 700, according to one embodiment of the invention, is shown. In the distal segment (toward the patient), the system 700 may terminate in a male luer connector 710. Upstream from the male luer connector 710, the system 700 may comprise various components connected with a fluid line comprising single lumen tubing. In order from closest to the male luer connector 710 to farthest from the male luer connector 710, the system 700 may comprise a stopcock valve 720, a sampling site 730, a pressure transducer assembly 740 including a dynamic restrictor 745, another sampling site 750, a reservoir 760, a pull-tab and restrictor assembly 770, and an IV assembly 780. The number of sampling sites does not limit the invention. In different embodiments, either one or both of the sampling sites 730, 750 may be present.

The length of tubing between the male luer connector 710 and the pressure transducer assembly 740 may be fixed, and may be approximately 40 inches. Since the length of tubing between the male luer connector 710 and the pressure transducer assembly 740 is fixed, a hardware filter 741 may be included in the pressure transducer assembly 740 (e.g., at a cable connector) to remove all distortions. With the assistance of the hardware filter 741, soft and compliant material may be used for the tubing between the male luer connector 710 and the pressure transducer assembly 740. Therefore, the pressure transducer assembly 740 may be mounted inline at a fixed distance from the patient. In one embodiment, the pressure transducer 740 may be fixed on the patient near the phlebostatic axis.

The dynamic restrictor 745 may serve two functions: 1) it isolates the pressure transducer from distortions due to the blood sampling portion of the system 700 (upstream from the pressure transducer assembly 740); and 2) it allows liquid flow when the system 700 is flushed, or when the blood sampling portion of the system is in use. Because the dynamic restrictor 745 allows the hydraulic connection between the two portions of the system for flushing and patency, but isolates the two portions from each other with respect to pressure transmission, soft and compliant material may be used for the tubing in the blood sampling portion of the system 700 without negatively affecting the accuracy of blood pressure measurements. Moreover, the length of the tubing in the blood sampling portion of the system 700 is completely flexible. In other words, longer tubing may be used in this portion.

It should be appreciated that the restrictor that allows hydraulic connection but isolates pressure transmission between the pressure monitoring portion and the blood sampling portion of the system (e.g., restrictor 350 of FIG. 3, restrictor between the pressure transducer 430 and the sampling site 442 in FIG. 4, restrictor in the check-valve/restrictor assembly 535 of FIG. 5, and dynamic restrictor 745 in FIG. 7, etc.) may be of any suitable structure, form, etc. Several example structures of the dynamic restrictor will be described in detail below. However, the particular structure or implementation of the dynamic restrictors described do not in any way limit the embodiments of the invention.

Referring to FIGS. 8A and 8B, diagrams illustrating cross-section views of an example restrictor 800 are shown. The restrictor 800 may comprise a plurality of elastic flaps 810 within a round frame 820. It should be appreciated that the shape of the frame does not limit the invention. The edges of neighboring elastic flaps 810 are spaced by slits 830. The elastic flaps 810 may be made with any suitable elastic material, such as an elastomer. When liquid (e.g., blood and/or saline) comes into contact with the face of the elastic flaps 810, liquid pressure may force the elastic flaps 810 to deflect backwards, thus further opening up the slits 830 between the elastic flaps 810 and allowing the liquid to flow through the restrictor 800. In one embodiment, the slits 830 may allow approximately 3 mL/hr of saline to flow through for live patency. It should be appreciated that energy is absorbed in opening the elastic flaps 810. The resonant energy from the blood sampling portion may be small relative to energy required to deflect the elastic flaps 810. Therefore, the resonant energy from the blood sampling portion may be absorbed by the restrictor 800 and pressure transmission reduced or isolated. In different embodiments, a single restrictor 800 may be used, or a plurality of restrictors 800 may be used and placed in series. With a proper combination of size geometry, elasticity and/or thickness of the flaps, and quantity of the restrictors 800, the pressure transducer may be isolated from the resonance of the blood sampling portion, while the pressure drop across the restrictor 800 is not prohibitively increased.

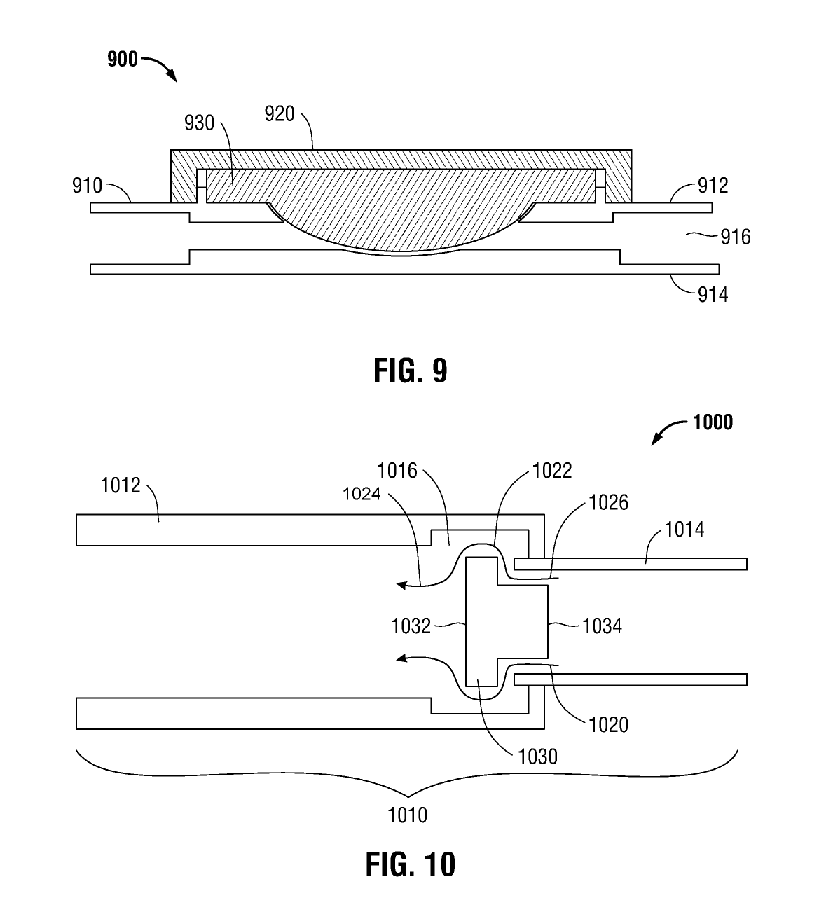

Referring to FIG. 9, a diagram illustrating a cross-section view of another example restrictor 900 is shown. The restrictor 900 is a restrictor that is capable of bi-directional pressure assisted opening. The restrictor 900 comprises a housing 910, a cap 920, and a diaphragm 930. The housing 910 and the cap 920 may be made of plastic. The diaphragm 930 may be an elastomeric diaphragm. The diaphragm 930 may be round in shape and may have a flat rim and a convex center. The diaphragm 930 may be held in place with its flat rim sandwiched between the cap 920 and a first side 912 of the housing 910. The housing 910 has a through portion 916 between the first side 912 and a second side 914 that allows liquid to pass through. Further, the housing 910 has a cut-out portion on the first side 912, allowing the convex center of the diaphragm 930 to extend into the through portion 916 of the housing 910. In a quiescent state, a small gap that allows a small flow of liquid to pass through may exist between the convex center of the diaphragm 930 and the inner face of the second side 914 of the housing 910. In one embodiment, the restrictor 900 may allow an approximately 3 mL/hr flow through it.

When liquid with a sufficient pressure within the through portion 916 of the housing 910 comes into contact with the convex center of the diaphragm 930, the convex center of the diaphragm 930 may deflect inwards under the pressure of the liquid, allowing more liquid to pass through. In one embodiment, when the liquid is pressurized to approximately or above 4 to 5 pounds per square inch (psi), the diaphragm 930 may open further (e.g., deflecting inwards), allowing an approximately 1-3 milliliters/second (mL/s) flow to pass through for pressures on the order of 4-20 psi.

Referring to FIG. 10, a diagram illustrating a cross-section view of yet another example restrictor 1000 is shown. The housing 1010 of the restrictor 1000 comprises a hollow first portion 1012 (illustrated on the left side) and a hollow second portion 1014 (illustrated on the right side). At or near the location where the two portions 1012, 1014 meet, the first portion 1012 further comprises a cut-out portion 1016 on the inside that is so shaped as to retain an elastic web 1020 that has a bulging middle portion 1022 and two narrow end portions 1024, 1026. An inelastic T-shaped block 1030 may be deposited within the elastic web 1020. The T-shaped block 1030 may comprise a plate portion 1032 (corresponding to the horizontal bar in the letter T) and a column portion 1034 (corresponding to the vertical bar in the letter T). The T-shaped block 1030 is so shaped that the plate portion 1032 may move in the horizontal direction as illustrated inside the elastic web 1020 between the two ends of the cut-out portion 1016 of the first portion 1012 of the housing 1010, subject to the elastic forces of the elastic web 1020, while the column portion 1034 may extend into the inside of the second portion 1014 of the housing 1010. In a quiescent state, the plate portion 1032 of the T-shaped block 1030 may be retained in the bulging middle portion 1022 of the elastic web 1020, and the middle portion 1022 of the elastic web 1020 may be in contact with the end of the cut-out portion 1016 closer to the second portion 1014, or with a protruding portion of the second portion 1014 near the end of the cut-out portion 1016, as illustrated. Therefore, the middle portion 1022 prevents liquid from passing through on the outside of the elastic web 1020 while in the quiescent state. Further, the internal diameter of the middle portion 1022 of the elastic web 1020 may be slightly greater than the diameter of the plate portion 1032 of the T-shaped block 1030, and the internal diameter of the end portion 1026 of the elastic web 1020 may be slightly greater than the diameter of the column portion 1034 of the T-shaped block 1030. Therefore, in the quiescent state, a gap exists between the inside of the elastic web 1020 and the T-shaped block 1030, allowing a flow with a pressure lower than a threshold to pass through the elastic web 1020. In other words, when the flow pressure is below a threshold, the liquid may pass through the restrictor 1000.

If there is a sufficient liquid pressure coming from the direction of the second portion 1014 (e.g., during flushing), the T-shaped block 1030 may be pushed in the direction from the second portion 1014 to the first portion 1012. Accordingly, the plate portion 1032 may move into the end portion 1024 of the elastic web 1020 (but the plate portion 1032 cannot move past the end of the cut-out portion 1016 of the first portion 1012, as illustrated), flattening the middle portion 1022. Therefore, a greater flow of the liquid may pass through the restrictor 1000 via the space between the outside of the elastic web 1020 and the inside of the cut-out portion 1016. Conversely, if there is a sufficient liquid pressure coming from the direction of the first portion 1012, the T-shaped block 1030 may be pushed in the direction from the first portion 1012 to the second portion 1014. Accordingly, the T-shaped block 1030 may be pushed tightly against the end of the cut-out portion 1016 closer to the second portion 1014, or against a protruding portion of the second portion 1014 near the end of the cut-out portion 1016, as illustrated, at the underside of the plate portion 1032 (e.g., the side of the plate portion 1032 where the column portion 1034 is located), closing off the channel inside the elastic web 1020 that would allow liquid to flow through. Therefore, if the flow pressure is above a threshold and is coming from the direction of the first portion 1012, the flow may be prevented from passing through the restrictor 1000.

Therefore, embodiments of the invention relate to an integrated blood sampling-pressure monitoring system, comprising: a pressure transducer; a blood sampling portion; and a restrictor interposed between the pressure transducer and the blood sampling portion, wherein the pressure transducer is located closer to a patient than the blood sampling portion along a fluid line when the blood sampling-pressure monitoring system is connected to the patient, and wherein the restrictor allows a fluid connection between the pressure transducer and the blood sampling portion for flushing and patency, but isolates the pressure transducer from the blood sampling portion with respect to pressure transmission. Compared to a known, existing integrated blood sampling-pressure monitoring system, embodiments disclosed herein may help reduce or remove the distortions to the blood pressure waveform caused by the blood sampling portion of the system, thus improving the accuracy of blood pressure measurements. The length of the tubing for the portion of the system downstream from the pressure transducer may be fixed for all kits, potentially enabling manufactures to benefit from economies of scale. The pressure transduce assembly may further comprise a hardware filter that removes all distortions. The restrictor and the hardware filter may further allow longer, softer, and/or more compliant tubing to be used, increasing the flexibility of the system.

The various illustrative logical blocks, processors, modules, and circuitry described in connection with the embodiments disclosed herein may be implemented or performed with a general purpose processor, a specialized processor, circuitry, a microcontroller, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor may be a microprocessor or any conventional processor, controller, microcontroller, circuitry, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module/firmware executed by a processor, or any combination thereof. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. An integrated blood sampling-pressure monitoring system to sample blood from a patient and to measure blood pressure of the patient, comprising:
a pressure transducer to measure the blood pressure of the patient;
a blood sampling portion to sample blood from the patient; and
a restrictor interposed between the pressure transducer and the blood sampling portion, the pressure transducer being located closer to the patient than the blood sampling portion along a fluid line when the blood sampling-pressure monitoring system is connected to the patient.

2. The integrated blood sampling-pressure monitoring system of claim 1, wherein the restrictor allows a fluid connection between the pressure transducer and the blood sampling portion while isolating the pressure transducer from the blood sampling portion with respect to pressure transmission.

3. The integrated blood sampling-pressure monitoring system of claim 1, wherein the pressure transducer is a disposable pressure transducer.

4. The integrated blood sampling-pressure monitoring system of claim 1, wherein the blood sampling portion comprises a sampling site, a stopcock valve, and a reservoir.

5. The integrated blood sampling-pressure monitoring system of claim 1, wherein the pressure transducer is part of a pressure transducer assembly that further comprises a hardware filter that removes pressure waveform distortions.

6. The integrated blood sampling-pressure monitoring system of claim 1, wherein the pressure transducer is fixed to an arm of the patient and positioned at approximately a same height as a phlebostatic axis of the patient.

7. The integrated blood sampling-pressure monitoring system of claim 1, wherein the restrictor comprises a check-valve restrictor assembly or a dynamic restrictor.

8. A method for implementing an integrated blood sampling-pressure monitoring system to sample blood from a patient and to measure blood pressure of the patient, comprising:
providing a pressure transducer to measure the blood pressure of the patient;
providing a blood sampling portion to sample blood from the patient; and
providing a restrictor interposed between the pressure transducer and the blood sampling portion, the pressure transducer being located closer to the patient than the blood sampling portion along a fluid line when the blood sampling-pressure monitoring system is connected to the patient.

9. The method of claim 8, wherein the restrictor allows a fluid connection between the pressure transducer and the blood sampling portion while isolating the pressure transducer from the blood sampling portion with respect to pressure transmission.

10. The method of claim 8, wherein the pressure transducer is a disposable pressure transducer.

11. The method of claim 8, wherein the blood sampling portion comprises a sampling site, a stopcock valve, and a reservoir.

12. The method of claim 8, wherein the pressure transducer is part of a pressure transducer assembly that further comprises a hardware filter that removes pressure waveform distortions.

13. The method of claim 8, wherein the pressure transducer is fixed to an arm of the patient and positioned at approximately a same height as a phlebostatic axis of the patient.

14. The method of claim 8, wherein the restrictor comprises a check-valve restrictor assembly or a dynamic restrictor.

15. A blood pressure measurement system for an integrated blood sampling-pressure monitoring system that includes a blood sampling portion to sample blood from a patient and that measures a patient's blood pressure, the blood pressure measurement system comprising:
a pressure transducer to measure the blood pressure of the patient; and
a restrictor coupled to the pressure transducer, wherein the patient, the pressure transducer, and the blood sampling portion are connected by a fluid line and the restrictor is interposed between the pressure transducer and the blood sampling portion along the fluid line, the pressure transducer being located closer to the patient than the blood sampling portion along the fluid line when the blood sampling-pressure monitoring system is connected to the patient.

16. The blood pressure measurement system of claim 15, wherein the restrictor allows a fluid connection between the pressure transducer and the blood sampling portion while isolating the pressure transducer from the blood sampling portion with respect to pressure transmission.

17. The blood pressure measurement system of claim 15, wherein the pressure transducer is a disposable pressure transducer.

18. The blood pressure measurement system of claim 15, wherein the blood sampling portion comprises a sampling site, a stopcock valve, and a reservoir.

19. The blood pressure measurement system of claim 15, wherein the pressure transducer is part of a pressure transducer assembly that further comprises a hardware filter that removes pressure waveform distortions.

20. The blood pressure measurement system of claim 15, wherein the pressure transducer is fixed to an arm of the patient and positioned at approximately a same height as a phlebostatic axis of the patient.

21. The blood pressure measurement system of claim 15, wherein the restrictor comprises a check-valve restrictor assembly or a dynamic restrictor.
